(19) United States
(12) Patent Application Publication
(10) Pub. No.: US 2022/0395192 A1
(43) Pub. Date: Dec. 15, 2022

(54) MOBILE AND NON-INTRUSIVE DEVICE FOR SLEEP APNEA SCREENING AND TELEMEDICINE

(71) Applicant: Duke University, Durham, NC (US)

(72) Inventors: Hau-Tieng Wu, Durham, NC (US); Uei-Ming Jow, Durham, NC (US); Cheng-Yao Chen, Durham, NC (US)

(21) Appl. No.: 17/840,773

(22) Filed: Jun. 15, 2022

Related U.S. Application Data
(60) Provisional application No. 63/210,725, filed on Jun. 15, 2021.

(51) Int. Cl. A61B 5/08; A61B 5/00

(52) U.S. Cl. CPC A61B 5/0826; A61B 5/7475; A61B 5/7221; A61B 5/6823; A61B 2562/0219

(57) ABSTRACT

Systems and methods for monitoring sleep using a sleep monitoring system includes a first component and a second component. The first component is configured to be coupled to a chest of the subject and the second component is configured to be concurrently coupled to an abdomen of the subject. Each component includes a housing, a pair of electrode pads mounted on an underside of the respective housing, and an ECG sensor circuit communicatively coupled to the respective pair of electrode pads. The first component further includes a photoplethysmogram sensor that includes at least one light source and at least one photodetector mounted on the underside of the housing of the first component at a location between the first pair of electrode pads.

### RELATED APPLICATIONS

This application claims the priority benefit of U.S. Provisional Patent Application No. 63/210,725, filed Jun. 15, 2021, entitled “MOBILE AND NON-INTRUSIVE DEVICE FOR SLEEP APNEA SCREENING AND TELEMEDICINE,” the entire contents of which are hereby incorporated herein by reference.

### BACKGROUND

The present invention relates to systems and methods for monitoring and tracking biometric data. More specifically, in some implementations, the present invention relates to systems and methods for monitoring biometric data for human subjects that have been diagnosed with or are suspected to have sleep apnea and for detecting sleep apnea related events.

### SUMMARY

Sleep apnea is one of the most common sleep disorders, affecting both children and adults. It is marked by abnormal breathing and can lead to potentially serious health consequences. Currently, patient monitoring is performed in a clinical setting in order to accurately capture all of the related biomarkers. However, the equipment used for polysomnography (PSG) is large, cumbersome, and resource heavy. Additionally, the patient is typically connected to the equipment through multiple wires and sensors, which can interfere with sleep and, therefore, undermine the purpose of the intervention. Further, the data analysis of a single sleep apnea episode is a labor-intensive work, which takes significant time (2-3 hours for a trained expert). As a result, sleep laboratories are uncommon, expensive, and often have long wait times.

There are some at-home monitoring solutions, but these systems sacrifice accuracy and capability for portability. Thus, there is an ongoing need for improved systems and methods for accurate, fully-featured, and portable sleep apnea monitoring.

In some implementations, the system and methods described herein provide a sleep monitoring device comprising, consisting of, or consisting essentially of a first component configured to be attached to a chest of a subject and a second component configured to be attached to an abdomen of the subject and in electronic communication with the first component. The first component comprises one or more photoplethysmogram (PPG) sensors, an electrocardiogram sensor, an electrical impedance plethysmography sensor, and an inertial measurement unit sensor. The second component comprises an electrocardiogram sensor, an electrical impedance plethysmography sensor, and an inertial measurement unit sensor. In some implementations, the first and/or second components further include one or more controllers, one or more memories, one or more wireless communication chips, one or more antennas, one or more electrodes, one or more temperature sensors, one or more pressure sensors, one or more moisture sensors, and one or more power sources.

In some implementations, the system further includes a remote computing device configured to receive data from the first and/or second components and to perform one or more of the following functions: (1) an oxygen level evaluation configured to convert multi-channel PPG signals recorded from the chest to an oxygen level, (2) a respiratory effort evaluation configured to convert tri-axial acceleration signals into abdominal and thoracic movement signals that match those recorded from respiratory inductance plethysmography, (3) a signal quality optimization that maximizes utilization of available signals, and/or a report of apnea-hypopnea index (AHI) and oxygen desaturation index.

In some implementations, the first component, the second component, and/or the remote computing device are configured to determine a start time, an end time, and a type of a sleep apnea event based on one or more signals measured and recorded by the system (e.g., a respiratory signal). In some implementations, the first component and/or the second component further includes a user interface for manually logging an “event.” In some implementations, the first component and the second component are synchronized to have a variation of the differences of less than 100 ms.

In one embodiment, the invention provides a sleep monitoring system comprising a first component and a second component. The first component is configured to be coupled to a chest of the subject and the second component is configured to be concurrently coupled to an abdomen of the subject. Each component includes a housing, a pair of electrode pads mounted on an underside of the respective housing, and an ECG sensor circuit communicatively coupled to the respective pair of electrode pads. The first component further includes a photoplethysmogram sensor that includes at least one light source and at least one photodetector mounted on the underside of the housing of the first component at a location between the first pair of electrode pads.

In some implementations, the first ECG sensor circuit and the second ECG sensor circuit are time synchronized by a clock signal transmitted from the second component to the first component through a wired communication interface. In some implementations, the first component further includes an electrical impedance plethysmogram sensor circuit communicatively coupled to at least one electrode pad of the first pair of electrode pads and at least one electrode pad of the second pair of electrode pads.

In another embodiment, the invention provides a method of calculating a biometric using a signal surrogate mechanism. A primary data signal and a secondary data signal are defined for a first biometric. A signal quality of the primary data signal is evaluated and the first biometric is calculated based on the primary data signal in response to determining that the signal quality of the primary data signal satisfies one or more signal quality criterion for the primary data signal. However, the first biometric is calculated based on the secondary data signal in response to determining that the signal quality of the primary data signal does not satisfy the one or more signal quality criterion for the primary data signal and that the signal quality of the secondary data signal satisfies one or more signal quality criterion for the secondary data signal.

Other aspects of the invention will become apparent by consideration of the detailed description and accompanying drawings.

### BRIEF DESCRIPTION OF THE DRAWINGS

FIG. 1 is a schematic diagram of a dual-component sleep apnea monitoring device positioned on a body of a human subject in accordance with one embodiment.

FIG. 2 is an overhead view of a first component (i.e., a chest device housing) of the dual-component sleep apnea monitoring device of FIG. 1.

FIG. 3 is an overhead view of a second component (i.e., an abdomen device housing) of the dual-component sleep apnea monitoring device of FIG. 1.

FIG. 4A is a schematic block diagram the first component (i.e., the chest device housing) of the dual-component sleep apnea monitoring device of FIG. 1.

FIG. 4B is a schematic block diagram the second component (i.e., the abdomen device housing) of the dual-component sleep apnea monitoring device of FIG. 1.

FIG. 5 is a flowchart of a method of verifying signal quality upon start-up followed by data monitoring performed by the dual-component sleep apnea monitoring device of FIG. 1.

FIG. 6 is a flowchart of a signal surrogate method performed by the dual-component sleep apnea monitoring device of FIG. 1 used to determine various metrics.

FIG. 7 is a flowchart of another example of a signal surrogate method performed by the dual-component sleep apnea monitoring device of FIG. 1.

FIG. 8 is a flowchart of a method for detecting sleep apnea events performed by the dual-component sleep apnea monitoring device of FIG. 1.

FIG. 9 is a flowchart of a method performed by the dual-component sleep apnea monitoring device of FIG. 1 for verifying signal quality, calculating metrics, and detecting sleep apnea events.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways.

Although sleep apnea is a common condition, the ability to understand each patient's condition remains complicated. Some existing systems for PSG are expensive, bulky, and unsuitable for large-scale screening and homecare. Simplified PSG systems, which can be used at home, may be less accurate due to a lack of a controlled recording environment and medical expert oversight. Existing PSG systems are also complicated and can interfere with sleep. Therefore, continuous at-home sleep monitoring is not currently feasible with existing systems. A light, robust, easy-to-use, and non-intrusive multi-sensor device, such as illustrated in the various examples below, is needed for a large-scale sleep apnea screening and sleep quality evaluation in the digital health era. Additionally, both medical doctors and patients benefits from a balance between data accuracy and convenience for home-based systems. The systems and methods described in the examples below address these and other challenges by providing a low-profile, multi-sensor sleep monitoring system.

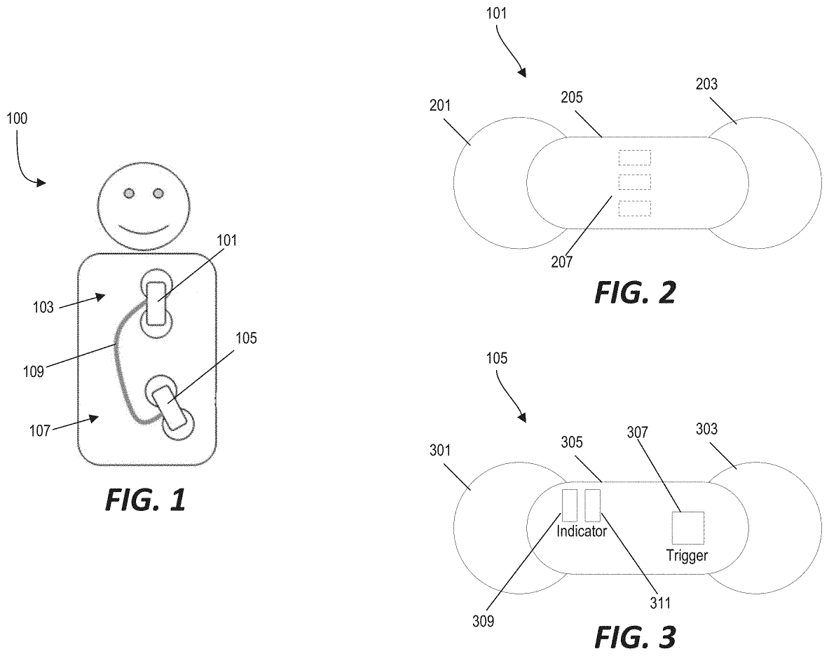

FIG. 1 illustrates an example of a sleep apnea monitoring system positioned on a patient 100. The system includes a first component 101 positioned on a chest 103 of the patient 100 and a second component 105 positioned on an abdomen 107 of the patient 100. The first component 101 and the second component 105 are coupled by a cable 109 configured to transfer electrical power and/or data between the first component 101 and the second component 105 during operation of the system.

As illustrated in further detail in FIG. 2, the first component 101 includes a first electrode pad 201 and a second electrode pad 203 coupled to each other by a chest device housing 205. The electrode pads 201, 203 can be attached to the chest 103 of the patient 101, for example, using skin adhesives. A photoplethysmogram (PPG) sensor system 207 is positioned on an underside of the chest device housing 205 between the first electrode pad 201 and the second electrode pad 203. The PPG sensor system 207 includes one or more LEDs and one or more light sensors and is configured to sense, for example, blood oxygen level of the patient 100 while the first component 101 is coupled to the chest 103 of the patient 100.

As illustrated in further detail in FIG. 3, the second component 103 includes a third electrode pad 301 and a fourth electrode pad 303 coupled to each other by an abdomen device housing 305. The electrode pads 301, 303 can be selectively attached to the abdomen 107 of the patient 100 as illustrated in FIG. 1, for example, using skin adhesives. A “trigger” button 307 is positioned on a top side of the abdomen device housing 305. A user will depress the trigger button 307 to “tag” various specific events or times during a sleep episode that might be relevant to sleep apnea monitoring. For example, a subject can use the trigger button 307 to tag occurrences of waking periods, coughing, bathroom trips, feelings of nausea, etc. These tags are recorded to memory with a time stamp and, thereby, the system can identify other biometer data (e.g., heart rate, ECG, etc.) corresponding to the user-tagged event. These time-stamped “tags” can also serve as “memory-joggers” in subject interviews after the monitoring period.

The top side of the abdomen device housing 305 also includes a series of LED indicators 309, 311 operable to communicate information to a user regarding the operating status of the system (e.g., whether the components 101, 103 are attached properly, whether sensed signals are of sufficient quality, etc.). In some embodiments, the electrode pads 201, 203, 301, 303 are removable and/or replaceable.

In other implementations, the top side of the abdomen device housing 305 and/or the chest device housing 205 may include other user interface features. For example, in some implementations, the abdomen device housing 305 and/or the chest device housing 205 may includes a power (on/off) button and/or a button to begin a recording session. Additionally or alternatively, the abdomen device housing 305 and/or the chest device housing 205 may include an emergency button to perform suitable functions such as shutting off the device, contacting emergency medical help, etc. Further, in some implementations, the user interface can include indicators that communicate system information such as power, data transfer, and data characteristics, including the aforementioned “smart light” (e.g., LEDs 309, 311).

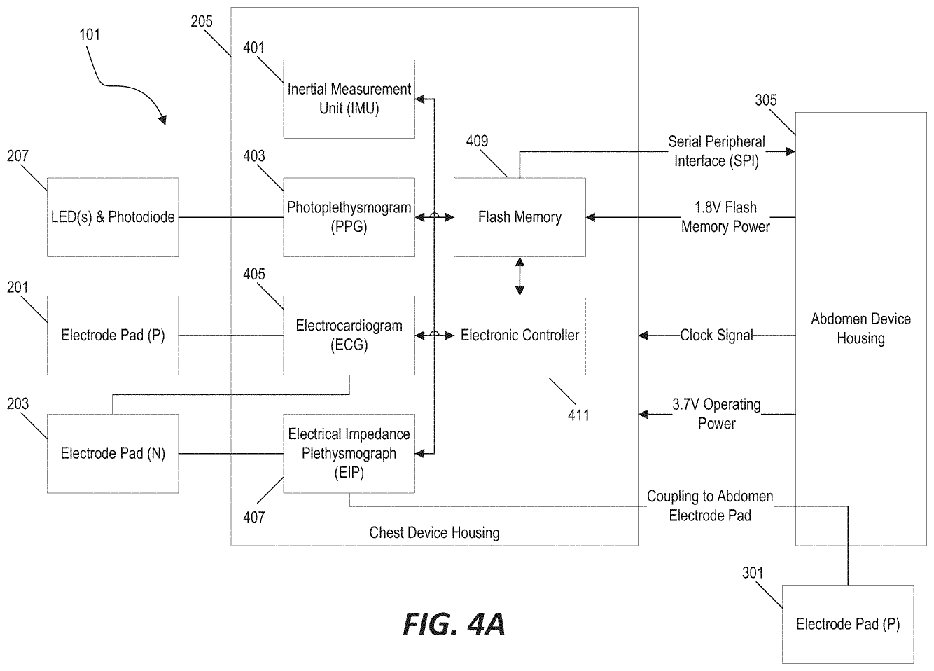

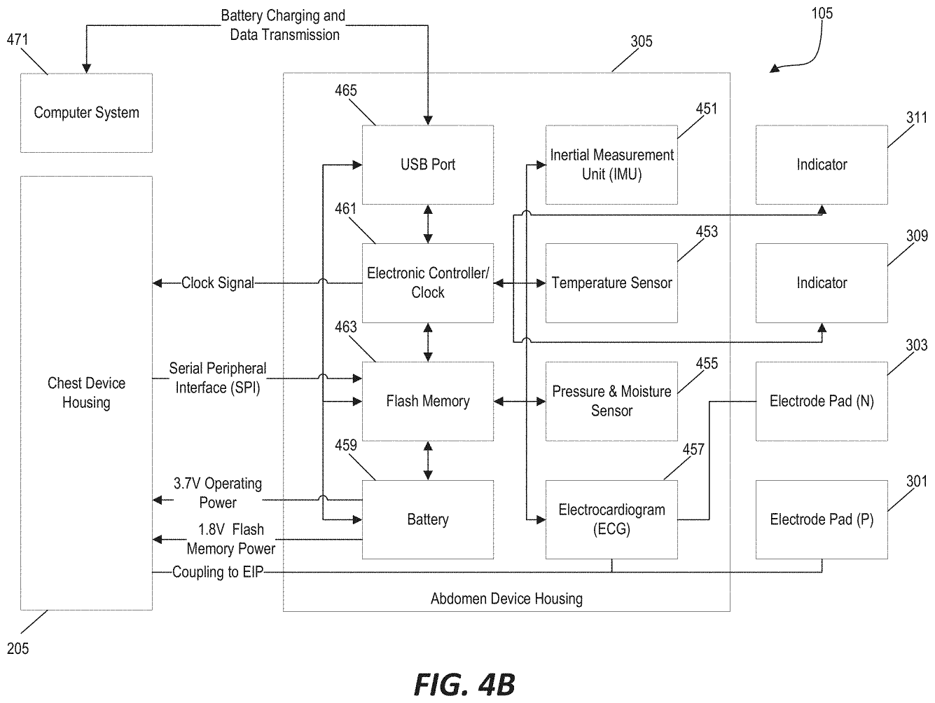

FIGS. 4A and 4B illustrate the internal components and operation of the first component 101 and the second component 103, respectively. As illustrated in FIG. 4A, the chest device housing 205 includes internal circuitry for an inertial measurement unit (IMU) 401, a photoplethysmogram (PPG) unit 403, an electrocardiogram (ECG) unit 405, and an electrical impedance plethysmograph (EIP) unit 407. In some implementations, the IMU 401 includes a tri-axial accelerometer and a tri-axial gyroscopic sensor and is configured to generate an output signal indicative of movement of the chest device housing 205 (e.g., movements caused by movements of the chest 103 of the patient 100 due to breathing). The PPG unit 403 is communicative coupled to the LED(s) and light sensor(s) of the PPG sensor system 207 and, in some implementations, operates as a pulse oximeter by illuminating the skin and then measuring changes in light absorption. The ECG unit 405 is communicatively coupled to the first electrode pad 201 and the second electrode pad 203 and operates to measure an electrocardiogram of the patient 100. The EIP unit 407 is communicatively coupled to the second electrode pad 203 and is also coupled, through the abdomen device housing 305 to the third electrode pad 301.

In some implementations, each measurement unit (e.g., the IMU 401, the PPG unit 403, the ECG unit 405, and the EIP unit 407) are provided as separate electronic components configured to generate a biometric data signal based on the sensors and/or electrodes communicatively coupled thereto. In other implementations, the measurement units may be implemented as circuitry on a single circuit board mounted within the chest device housing 205. As illustrated in FIG. 4A, the chest device housing 205 also includes a non-transitory, computer-readable memory (e.g., flash memory 409) and, in some implementations, each measurement unit is configured to communicate directly with the flash memory 409 to store the sensed biometric signal data to the flash memory 409. In some implementations, the chest device housing 205 also includes an electronic controller 411 that may be configured, for example, to facilitate the storage of biometric data from the measurement units to the flash memory 409, to compute specific metrics based on the output signals from one or more of the measurement units, to evaluate the quality of the output signals from the measurement units, and/or to facilitate data communications with other systems and device (e.g., the abdomen device housing 305 and/or another external computing system).

As illustrated in FIG. 4B, the abdomen device housing 305 includes a second inertial measurement unit 451, a temperature sensor 453, a pressure/moisture sensor 455, and an ECG unit 457. The second IMU 451 is similarly configured to measure movements of the abdomen device housing 305 (e.g., caused by movements of the abdomen 107 of the patient 100 due to breathing). In some implementations, the temperature sensor 453 is positioned within the abdomen device housing 305 and configured to measure a body temperature of the patient 100 and/or an ambient air temperature. In some implementations, the temperature sensor 453 includes a separate temperature sensing device (e.g., a thermistor or a thermocouple) while, in other implementations, the temperature sensor 453 is coupled to the third electrode pad 301 and/or the fourth electrode pad 303 and configured to sense a body temperature using the electrode pad(s). The pressure/moisture sensor 455 is configured to measure the air pressure and humidity in the ambient environment. The second ECG unit 457 is communicatively coupled to the third electrode pad 301 and the fourth electrode pad 303 and operates to measure an electrocardiogram signal between the third electrode pad 301 and the fourth electrode pad 303.

The abdomen device housing 305 also includes a power source such as, for example, battery 459. The battery 459 is configured to provide operating power to the internal components of the abdomen device housing 305 and is also configured to provide power to the internal components of the chest device housing 301 (e.g., through the cable 109).

The abdomen device housing 305 includes an electronic controller 461 and a non-transitory, computer-readable memory (e.g., flash memory 463). The flash memory 463 is configured to store biometric data from the measurement units of the abdomen device housing 305 (e.g., the IMU 451, the temperature sensor 453, the pressure/moisture sensor 455, and the ECG unit 457). In some implementations, the measurement units are configured to interface directly with the flash memory 463 to store data thereto while, in other implementations, the electronic controller 461 may be configured to facilitate and manage data storage operations. In some implementations, the electronic controller 461 may be configured to receive/monitor output signals from one or more of the measurement units, calculate metrics based on the output signals, monitor/evaluate the quality of the output signals from the measurement units, and/or to facilitate communications between the abdomen device housing 305 and other computing systems. However, in other implementations, the electronic controller 461 may be a simple clock circuit that operates to synchronize data acquisition and storage between the abdomen device housing 305 and the chest device housing 205.

As illustrated in FIG. 4B, the abdomen device housing 305 also includes a USB port 465 that is selectively coupleable to an external power source and/or an external computer system 471 (e.g., a desktop computer, a laptop computer, a tablet computer, etc.). The battery 459 is charged by coupling the USB port to a power source or an external computer system 471 that is capable of transferring charging power to the USB port. The USB port is also configured to facilitate digital communication between an external computer system 471 and the electronic controller 461. In some implementations, the external computer system 471 is configured to read and write data directly to the flash memory 463.

FIGS. 4A and 4B also illustrate examples of data and power transfers between the chest device housing 205 and the abdomen device housing 305 through the cable 109. For example, the abdomen device housing 305 is configured to transmit a clock signal to the chest device housing 205 to enable the measurement units of the chest device housing 205 to synchronize data storage using the same clock signal as the measurement units of the abdomen device housing 305. The cable 109 also provides a serial peripheral interface (SPI) between the flash memory 409 of the chest device housing 205 and the flash memory 463 of the abdomen device housing 305 so that the biometric data recorded by the measurement units of the chest device housing 205 are transferred to and stored on the flash memory 463 of the abdomen device housing 305 for computing/analysis and/or to be further transmitted to the external computer system 471. Finally, in the example of FIGS. 4A and 4B, the chest device housing 205 does not have a separate power source (e.g., a battery). Instead, the cable 109 is configured to transfer 3.7v operating power from the battery 459 of the abdomen device housing 305 to the measurement units of the chest device housing 205 and to transfer 1.8V flash memory power from the battery 459 to the flash memory 409 of the chest device housing 205. Furthermore, in implementations where the chest device housing 205 includes a separate electronic controller 411, the cable 109 is configured to transfer operating power of an appropriate voltage from the battery 459 to the electronic controller 411. Although the example of FIGS. 4A and 4B illustrate a battery 459 located in the abdomen device housing 305, but no battery located within the chest device housing 205, in some other implementations, one or more batteries may be located within both the abdomen device housing 305 and the chest device housing 205. Furthermore, in some implementations where a single battery/power source is shared by both the first component 101 and the second component 105, that battery/power source may be positioned within the chest device housing 205 instead of the abdomen device housing 305.

In the examples described above, data communications between the chest device housing 205 and the abdomen device housing 305 are facilitated by a wired communication interface (e.g., cable 109). However, in some implementations, this wired communication interface may be replaced with a wireless communication interface. Furthermore, in some implementations, separate batteries may be included in both the chest device housing 205 and the abdomen device housing 305 such that the cable 109 can be omitted entirely. Similarly, in the examples described above, the abdomen device housing 305 is communicatively coupled to the external computer system 471 through the USB port (i.e., a wired communication interface). However, in some implementations, the abdomen device housing 305 may include a wireless communication device/antenna to facilitate wireless data communication with the external computer system 471. In various implementations, the wireless communication device may be provided instead of or in addition to the USB port.

These two components 101, 105 can be synchronized by wired or wireless link such that the variation of the time difference of these two devices is less than 100 ms. The two components 101, 105 are synchronized to record the vital signs such as electrocardiogram, blood oxygen level, motion, body temperature, pulse (heart rate), and breathing rate (respiratory rate) based on the output signals from the measurement units. The storage of the device can be accessed by various different methods including, for example, wireless transmission of data to a mobile device or storage in a cloud computing system. The data can be sent incrementally or saved on the device and downloaded after the monitoring period is complete.

In some implementations, the sleep monitoring system also includes a remote device (e.g., the remote computing system 471) configured to collect, store, and analyzing the collected data. The remote device can be in the form of hardware, software, or a combination thereof. In some implementations, the remote device is a computer, a tablet, or a mobile phone equipped with an algorithm. The remote device can collect the data from the wearable components from any suitable wired or wireless method, such as Bluetooth, WiFi, BLE, Zigbee, Z-Wave, 6LoWPAN, NFC, WiFi Direct, GSM, LTE, LoRa, NB-IoT, and LTE-M, 5G, etc. After collecting the data, the remote device analyzes the data. In some implementations, the data can optionally be sent to a centralized data storage center (e.g., “the cloud”), with or without the remote device, for analysis, storage, and retrieval. In some implementations, the remote device can be further configured to collect and incorporate additional relevant data from other sources, such as the environmental sound, subject's sound, ambient light and temperature, etc.

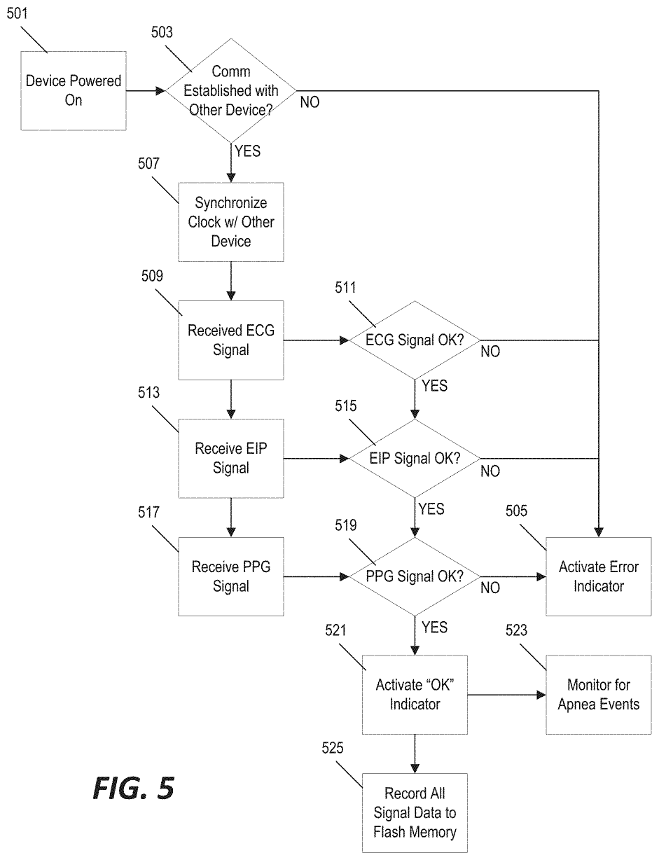

FIGS. 5 through 9 illustrates examples of methods performed by the system of FIG. 1 related to monitoring biometric data and/or detecting sleep apnea events. As discussed above in reference to FIGS. 4A and 4B, in some implementations, both the chest device housing 205 and the abdomen device housing 305 each include a separate internal electronic controller 411, 461 while, in other implementations, the abdomen device housing 305 includes an electronic controller 461, but the chest device housing 205 does not include a separate electronic controller 461. In other implementations, the chest device housing 205 may include an electronic controller 411, but the abdomen device housing 305 does not. And, in still other implementations, neither the chest device housing 205, nor the abdomen device housing 305 includes an electronic controller. Accordingly, in various different implementations, the methods illustrated in the examples of FIGS. 5 through 9 may be executed by the external computer system 471, the electronic controller 411, the electronic controller 461, or by coordinated combinations thereof. Unless specified otherwise, use of the phrase “controller” in the examples below is intended to refer to any individual controller 411, 461, 471 or combinations thereof.

In some implementations, the system is configured to monitor signal quality of the data captured by the measurement units and/or to initiate and monitor time synchronization between the measurement units of the first component 101 and the measurement units of the second component 103. The system may be configured to perform signal quality and time synchronization checks at start-up of the system (e.g., at the beginning of a sleep monitoring period) and/or periodically during operation of the system. In some implementations, when the system is activated, the controller will check the sensors/measurement units within the first 30 seconds after powering on and after receiving signals. If there is no signal, or if the signal quality is under a prescribed threshold, the indicator will display the information (e.g., on a graphical user interface of the external computer system and/or by activating one or both of the indicators 309, 311) so that the subject can adjust the device and restart the procedure. Multi-channel signals on each device are cross-checked by the dynamic range and frequency for fault detection. If a fault is detected, a calibration can be implemented. For additional data security, multi-device signals on each device can also be compared to ensure the signal quality.

FIG. 5 illustrates an example of a method executed by one or more controllers of the system (e.g., electronic controller 411, electronic controller 461, and/or the external computer system 471) upon start-up to initiate synchronization and to confirm the signal quality before beginning the monitoring operation. When the system/device(s) are first powered on (e.g., by a user pressing a “power” button on the user interface) (step 501), the controller determines whether communication is established between the first component 101 and the second component 105 (e.g., via the cable 109 or via a wireless communication interface) (step 503). In some implementations, this communication verification also includes determining whether communication is established between the external computer system 471 and one or both of the device components 101, 105. If communication between the device components cannot be established, then the controller generates a signal to activate an error indicator (e.g., indicator LED 309, 311) (step 505).

However, if communication between device components is properly established, then the controller synchronizes the clock between the first component 101 and the second component 105 (step 507). In some implementations, synchronization is established by the second component 105 generating and transmitting a “clock signal” to the first component 101 (as described above. In some implementations, proper time synchronization is verified by analyzing an incoming data signal to ensure that detected events align within a maximum defined synchronization tolerance. For example, in order to have an accurate measurement of heart rhythm, ECG signals from the two components should be synchronized and calibrated. The QRS complex of heart's rhythm is the combination of three of the graphical deflections seen on a typical ECG. In adults, the QRS complex normally lasts 80 to 100 ms. Therefore, the first and second component are preferably synchronized to have a time difference of less than 10 ms in order to capture an accurate waveform. This can be accomplished, for example, using the cable 109 linking the devices having a clock signal higher than 1 kHz. The cable 109 can also optionally be used for power, data transfer, and control actions. Alternately, it is possible to synchronize the two components using a wireless technology. The synchronization of the two components not only aligns the collected data for analysis but also dynamically enhances the signal-to-noise ratio and adjusts the signal drifting of specific channels.

Once proper time synchronization between the components 101, 105 is established and confirmed, the controller analyzes the quality of the output signals from the various measurement units to confirm proper operation of the measurement units and/or to confirm proper placement/adherence of the device components to the chest and abdomen of the human subject. In the example of FIG. 5, the controller receives the ECG signal from the ECG unit 405 and the ECG unit 457 (step 509) and analyzes each signal separately (and, in some implementations, a combination of the signals from each ECG unit 405, 457) to evaluate a quality of the ECG signal(s) (step 511). Similarly, the controller receives the output signal from the EIP unit 407 (step 513) and analyzes the signal to evaluate a quality of the EIP signal (step 515). Finally, the controller receives the output signal from the PPG unit 403 and analyzes the PPG signal to evaluate the quality of the PPG signal (step 519).

In various implementations, different types of algorithms may be applied by the controller to evaluate signal quality. For example, for signals where a characteristic waveform is expected (e.g., a periodic QRS complex of an ECG signal), the controller may be configured to monitor the signal to detect the shape of the QRS complex and a periodic repetition of similar waveforms. In some implementations, the controller may be configured to analyze each signal in combination with other related signals to confirm the quality of the captured data signal (e.g., by comparing the period/frequency of the ECG waveform with a pulse measured by the PPG signal and/or the EIP signal). The Applicant also notes that, although the example of FIG. 5 only illustrates signal verification for three signal types (i.e., ECG, PPG, and EIP), in other implementations, the controller may be configured to analyze a greater or lesser number of different signals and/or other types of signals in addition to or instead of those illustrated in the example of FIG. 5.

Some of the examples presented herein use the phrases “sufficient quality,” “insufficient quality,” “unsatisfactory quality,” etc. in discussing the evaluation of the data signals by the controller. In some implementations, the signal quality evaluation algorithm applied by the controller will be configured to produce a quantification of signal quality as either a numeric metric indicative of quality (e.g., how closely does the measured signal match the expected signal) and/or a binary determination of whether the measured signal satisfies one or more quality criterion. Accordingly, phrases such as “sufficient quality” used herein refer to a signal that satisfies one or more defined criterion for quality of the particular signal (e.g., a numeric quality metric exceeding a defined quality threshold) and phrases such as “insufficient quality” or “low quality” used herein refer to a signal that does not satisfied the one or more defined criterion for quality of the particular signal (e.g., a numeric quality metric that does not exceed the defined quality threshold for the signal).

If the controller determines that any one of the evaluated signals is of unsatisfactory quality, the controller will transmit a signal to activate and/or operate the error indicator (step 505). Conversely, if the controller determines that all of the signals are of sufficient quality, the controller will activate an “OK” indicator” to indicate to the user that the device components are properly positioned on the patient and that the signal quality is sufficient for sleep monitoring (step 521). The controller will then proceed to monitor the biometric data captured during the sleep period to detect apnea-related events (step 523) and will record the captured/measured biometric data to the flash memory of the device component(s) (step 525).

In the example of FIG. 5, the controller is configured to determine whether the quality of each data signal is sufficient or insufficient, and to output an error indication if any signal is determine to be of insufficient quality. However, for some biometrics, the same measurement can be quantified (independently or coordinately) based on the output for multiple different measurement units. For example, the heart rate of a patient can be determined based on the ECG signal or based on the PPG signal. Accordingly, in some implementations, the controller is configured to apply a signal surrogate mechanism to maximize the utilization of available signal so that the system can continue to operate even if one or more data signals are of lower quality. To accomplish this, the controller is configured by ranking the available data signal channels according to their functions. For example, for the respiratory effort, the tri-axial accelerator signal may be ranked first, and the electrocardiogram signal ranked second. For the heart rate, the ECG may be ranked first, and the photoplethysmogram ranked second. If the top-ranked channel is considered of low quality, the surrogate algorithm replaces or supplements the data of the top-ranked channel with data from the second-ranked channel. If both channels are considered of low quality, the segment is marked and not analyzed.

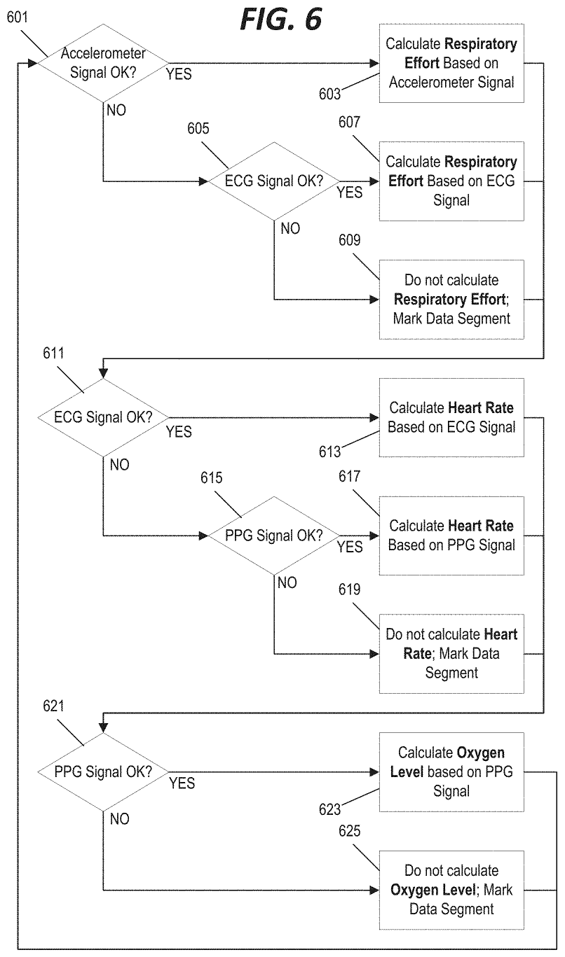

FIG. 6 illustrates an example of the signal surrogate mechanisms applied by the controller during monitoring of the patient's sleep cycle (e.g., after performing the method of FIG. 5). However, in some implementations, the method of FIG. 5 may be adapted to implement a signal surrogate mechanism as well (e.g., to determine whether the system can operate based on the available signal data even after determining that one or more specific signals are of lower quality). In the example of FIG. 6, the method is performed periodically throughout the monitoring period to determine whether the signal quality has changed (e.g., due to patient movement during sleep) and marks the recorded data stream(s) to indicate whether an alternative mechanism can or should be used to calculate various biometrics.

First, the controller analyzes the quality of the accelerometer signal from the IMU 401 and/or IMU 451 (step 601). If the accelerometer signals are of sufficient quality, then the controller will proceed to calculate the “respiratory effort” biometric based on the output signal of the IMU 401, 451 (step 603) (as discussed further below). Alternatively, if the quality of the IMU output signal is not of sufficient quality, then the controller will determine whether the ECG signal is of sufficient quality (step 605). If the accelerometer signal is not of sufficient quality, but the ECG signal is determined to be of sufficient quality, then the controller will calculate the “respiratory effort” biometric based on the ECG signal (step 607) (or, in some implementations, based on a combination of the available accelerometer signal and the available ECG signal). However, if the controller determines that both the accelerometer signal and the ECG signal are not of sufficient quality, then the controller will mark the data segment as lacking sufficient signal quality for calculation of the “respiratory effort” biometric and will not calculate respiratory effort (step 609) until the signal quality for one or both of the accelerometer signal and the ECG signal improves.

Similarly, for calculating a “heart rate” biometric, the controller will first determine whether the ECG signal is of sufficient quality (step 611) and, if so, the controller will calculate the “heart rate” biometric based on the ECG signal (step 613). If the ECG signal is determined to not be of sufficient quality, but the PPG signal is determined to be of sufficient quality (step 615), then the controller will calculate the “heart rate” biometric based on the PPG signal (step 617) (or, in some implementations, based on a combination of the available ECG signal and the available PPG signal). However, if the controller determines that both the ECG signal and the PPG signal are of insufficient quality, then the controller will mark the data segment as lacking sufficient signal quality for calculation of the “heart rate” biometric and will not calculate a heart rate (step 619) until the signal quality of one or both of the ECG signal and the PPG signal improves.

In some implementations, the controller may be configured to define a hierarchical “signal surrogate” for every metric monitored by the system. However, in other implementations, the controller may be configured to use the output from only a single measurement unit for calculating a particular biometric and, if that signal is not available or is not of sufficient quality, the controller will not attempt to calculate that biometric. For example, in the example of FIG. 6, the controller is configured to use only the PPG signal to calculate oxygen level of the patient. Accordingly, if the controller determines that the PPG signal is of sufficient quality (step 621), then the controller will calculate the “oxygen level” biometric based on the PPG signal. However, if the controller determines that the PPG signal is not of sufficient quality, then the controller will mark the data segment as lacking sufficient signal quality for calculation of the “oxygen level” biometric and will not calculate an oxygen level (step 625) until the signal quality of the PPG signal improves.

In some of the examples above, the output of the signal quality evaluation is a binary choice of “pass” or “fail” (i.e., the controller determines that the signal is of sufficient quality or is not of sufficient quality). However, in other implementations, the controller may be configured to apply the signal surrogate mechanism based on additional levels of determined signal quality. For example, the controller may be configured to determine whether a particular signal is of “high quality,” “low quality,” or “unusable quality.” In some implementations, a “low quality” signal might be insufficient for use in determining a biometric by itself, but can be used to calculate the biometric if the data of the “low quality” signal is supplemented by data from another “high quality” signal (e.g., “low quality” data from the IMU can be used to calculate respiratory effort” if supplemented by “high quality” data from the ECG). However, in some implementations, a signal of “unusable quality” cannot be used by the controller to calculate the biometer even if supplemented by data from another “high quality” signal.

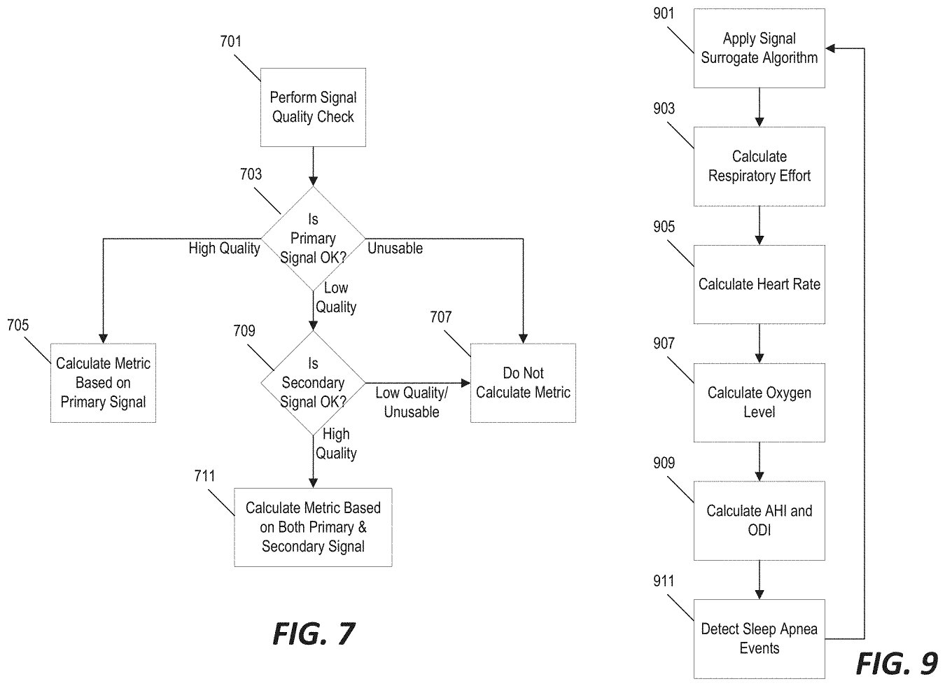

FIG. 7 illustrates an example of a signal surrogate method applied by the controller to determine whether a particular biometric can be calculated based on the available signal data. First, the controller performs the signal quality check on the applicable signals (step 611). If the primary signal (i.e., the highest ranked signal in the signal surrogate hierarchy for the particular biometric) is determined to be of “high quality” (step 703), then the controller calculates the biometric based on the primary signal (step 705). Conversely, if the primary signal is determined to be of “unusable quality” (step 703), then the controller determines that the biometric cannot be calculated and marks the data stream accordingly (step 707). However, if the controller assigns the intermediate signal quality classification (i.e., “low quality”) to the primary signal for the particular biometric (step 703), then the controller determines the signal quality of the secondary signal (step 709). If the primary signal is determined to be of low quality, but the second signal is determined to be of high quality, then the controller calculates the biometric based on the available primary signal data supplemented by the available secondary signal data (step 711). However, if the primary signal is determined to be of low quality and the quality of the secondary signal is determined to be anything less than “high quality” (i.e., low quality or unusable quality), then the controller determines that the biometric cannot be calculated and marks the data stream accordingly (step 707).

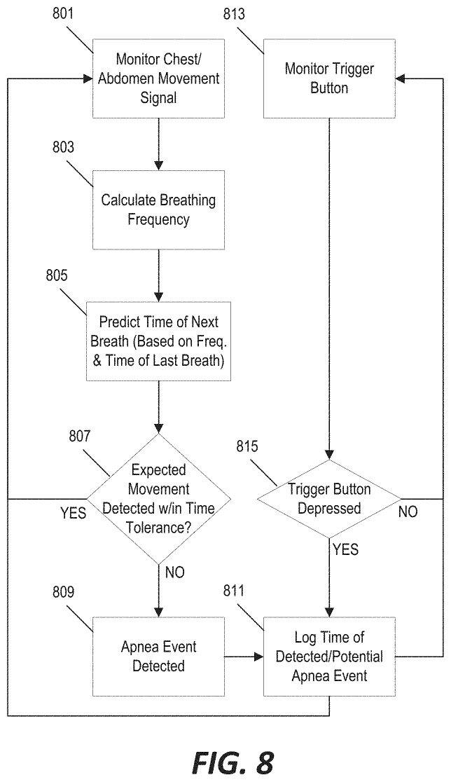

In addition to monitoring signal quality and calculating biometric data, in some implementations, the system is configured to detect “apnea events” based on the output signals from the various measurement units. One example of an apnea event is an occurrence in which regular breathing is disrupted during sleep by an obstructed airway. The quantity and frequency of such apnea events (as well as the corresponding biometrics recorded before, during, and after each apnea event) may be used by a medical professional in evaluating the patient's condition. FIG. 8 illustrates an example of a method executed by the controller for detecting and recording “apnea events” during sleep monitoring. During regular breathing, a patient's chest and abdomen will rise and fall in a generally periodic manner. Because the first component 101 is positioned on the chest 103 of the patient 100, regular breathing will cause the first component 101 to rise/fall and movement of the first component 101 can be monitored by the IMU 401 within the chest device housing 205. Similarly, because the second component 105 is positioned on the abdomen 107 of the patient 100, regular breathing will cause the second component 105 to rise/fall and movement of the second component 105 can be monitored by the IMU 451 within the abdomen device housing 305.

Accordingly, the controller monitors movement of the patient's chest 103 and abdomen 107 based on the output signal of the IMU 401 (chest) and the output signal of the IMU 451 (abdomen) (step 801). The controller calculates a breathing frequency (step 803) and, in some implementations, a breathing amplitude based on the output signals from IMU 401, 451. Based on the calculated breathing frequency and a detected time of the previous breath, the controller predicts a time of the next breath (step 805) and continues to monitor the output signals from the IMU 401, 451 to detect the actual time of the next breath. If the actual time of the next breath is determined to be within a determined “time tolerance” of the expected time of the next breath (step 807), then the controller determines that the patient is breathing normally and that no apnea event has occurred. However, if a next breath is not detected within the determine “time tolerance” of the expected time of the next breath (step 807), then the controller determines that the breathing pattern has been disrupted or altered and records the current time as a potential “apnea event” (step 809). The time of the potential apnea event is logged in the flash memory (step 811) and biometric data concurrent to the potential apnea event can be identified based on the time stamp of the potential apnea event for later analysis.

In addition to automatically detecting potential apnea events based on chest and/or abdomen movement, the second component 105 includes the trigger button 307 (as described above) through which the user can manually flag events that might be relevant to analysis of the biometric data. In some implementations, a medical professional may instruct the patient to press the trigger button each time that the patient awakes during the night due to what the patient perceives as an “apnea event.” Accordingly, in some implementations, the controller is configured to monitor the trigger button 307 (step 813) during the sleep monitoring period and, in response to determining that the trigger button 308 has been pressed (step 815), the controller logs the time of button press along with the times of the automatically detected apnea events (step 811).

To summarize operation of the system, FIG. 9 illustrates an example of the general operation of the system during a sleep monitoring period. The system periodically applies the signal surrogate algorithm to evaluate signal quality and to determine whether biometric data can be calculated based on the available signal(s) (step 901). In response to determining that the available signal data is of sufficient quality, the system then calculates respiratory effort (step 903), for example, based on the output signal from the IMU 401 (chest) and the IMU 451 (abdomen). The system also calculates a heart rate (step 905), for example, based on the ECG output signal and/or the PPG output signal and calculates an oxygen level (step 907) based on the PPG output signal. In some implementations, the system then proceeds to calculate an apnea-hypopnea index (AHI) and/or an oxygen desaturation index (ODI) based on the calculated/monitored biometrics during the sleep monitoring period (step 909). The system also operates to detect and log apnea events automatically and/or based on manually-identified “events” corresponding to a user pressing the trigger button 307 (step 911).

Although FIG. 9 shows these steps performed in sequences as a periodic loop, in various different implementations, the controller may be configured to perform each step at different frequencies. For example, in some implementations, the frequency at which the controller calculates biometrics such as respiratory effort, heart rate, and oxygen level may be greater than the frequency at which the controller applies the signal surrogate algorithm to evaluate the quality of the available data signals. Similarly, in some implementations, the controller may be configured to calculate some metrics periodically throughout the sleep monitoring period and the calculate other metrics only after completion of the sleep monitoring period. For example, in some implementations, the controller may be configured to respiratory effort, heart rate, and oxygen level periodically throughout the sleep monitoring period, but calculates AHI and ODI only after completion of the sleep monitoring period.

Furthermore, in some implementations, the system may be configured such that the first component 101 and the second component 105 are not coupled to the external computer system 471 during the sleep monitoring period and, instead, the second component 105 is coupled to the external computer system 471 after completion of the sleeping monitoring period so that the captured data can be uploaded to the external computing system 471 and further data analysis can be performed. Accordingly, in some implementations, some or all of the methods for analyzing and processing the captured data signals might be performed only after completion of the sleeping monitoring period when the second component 105 is communicatively coupled to the external computing system 471. For example, in some implementations, the sleep monitoring period is initiated by communicatively coupling the second component 105 to the external computing system and the start-up sequence of FIG. 5 is performed by the external computer system 471 based on data signals received by the external computer system 471 from the first component 101 and the second component 105. Then, after signal quality is confirmed (e.g., step 521 of FIG. 5), the external computer system 471 is disconnected from the second component 105. With the external computing system 471 disconnected, the first component 101 and the second component 105 operate to record time-series data from each measurement unit to the flash memories 409, 463 throughout the sleep monitoring period without performing any calculations or analysis on the captured data. After completion of the sleep monitoring period, the second component 105 is again communicatively coupled to the external computing system 471, all of the recorded time-series data is uploaded from the flash memories 409, 463 to the external computing system 471, and the external computing system 471 then proceed to process the collected data (e.g., applying the signal surrogate analysis, calculating the applicable biometric data, detecting/logging potential apnea events, etc.).

Similarly, in some implementations, the electronic controllers 411,461 of the first component 101 and/or the second component 105 may be configured to perform some data processing methods during the sleep monitoring period (e.g., less computationally intensive methods) and other data processing methods are performed by the external computing system 471 after the sleep monitoring period is completed and the second component 105 is coupled to the external computing system 471 for data upload. For example, in some implementations, a biometric may be calculated by the electronic controller 461 using a first method during the sleep monitoring period and, after completion of the sleep monitoring period, the same biometric maybe calculated by the external computing system 471 using a second method. The second method for calculating the biometric may be more computationally advanced and more accurate for use in post-monitoring analysis; however, by calculating the biometric during the sleep monitoring period using the simplified first method, the system is able to provide real-time indications of the biometric and/or adjust operation of the device during the sleep monitoring period based on the biometric.

In some implementations, the IMU 401 and/or the IMU 451 includes a 9-axial IMU with a 50 Hz sampling rate and 16-bit resolution. The 9-axes of the IMU 401, 451 include three-dimensional accelerometer, a three-dimensional gyroscopic sensor, and a three-dimensional magnetometer. In some implementations, the PPG unit 403 is configured to operate at a 100 Hz sampling rate with 16-bit resolution and is configured to monitor SpO2 and heart rate. In some implementations, the LED “pads” of the PPG sensor system 207 includes a larger size to allow for wavelength tuning adjustment. In some implementations, the ECG unit 405, 457 operate at a 500 Hz sampling rate with 16-bit resolution using positive and negative leads (e.g., electrode pads 201, 203 and electrode pads 301, 303). In some implementations, the EIP unit 407 operates at a 30 Hz sampling rate using a small current and sharing the at least some of the same electrode leads as the ECG units 405, 457. In some implementations, the temperature sensor 453 is a body temperature sensor configured to operate with a 1 Hz sampling rate and the pressure/moisture sensor 455 is configured to operate with a 0.1 Hz sampling rate.

In some implementations, the chest device housing 205 and the abdomen device housing 305 are configured to position the respective electrode pairs (i.e., electrode pads 201, 203 and electrode pads 301, 303) at a distance of between 40 mm and 60 mm from each other. In some implementations, the chest device housing 205 and the abdomen device housing 305 are sized to have a height of less than 5 mm and a with of less than 35 mm. In some implementations, the chest device housing 205 and the abdomen device housing 305 are the same size and shape while, in other implementations, the chest device housing 205 and the abdomen device housing 305 are differently shaped or sized. For example, in some implementations, the length of the chest device housing 205 is less than 70 mm (to account for the positioning of the PPG sensor system 207 between the electrode pads 201, 203) and the length of the abdomen device housing 305 is less than 50 mm.

In some implementations, the battery 459 is a rechargeable lithium ion battery will a battery life of at least 16 hours during sleep monitoring operation. In some implementations, the data synchronization in all channels (e.g., in every measurement unit of the first component 101 and the second component 105) has a latency of up to 0.1 ms. In some implementations, time synchronization between the measurement units of the first component 101 and the measurement units of the second component 105 results in a time difference of less than 100 ms. In some implementations, the USB port 465 is a USB 3.0 port.

Accordingly, the systems and methods described in the examples above provide a mobile and non-intrusive device for sleep apnea screening including a first component (to be placed on the chest during sleep monitoring) and a second component (to be placed on the abdomen during sleep monitoring). Each component includes a pair of electrode pads and are configured to record an ECG signal, an EIP signal, and a PPG signal. The system is further configured to calculate a plurality of biometrics based on the measured signals and to apply a signal surrogate mechanism to evaluate the quality of each signal and to determine whether deficient signal quality can be mitigated by calculating one or more biometrics using additional data from another signal. The system is also configured to detect and log potential apnea events in response to automatic event detection and in response to a user-indicated potential apnea event indicated by a user-activated trigger button.

The presently disclosed sleep device has a number of advantages over other systems. It is a simple, easy-to-operate system, where the heart rhythm and breathing patterns are determined by a simple set of patches. A novel arrangement of PPG sensors (including LED intensity and orientations) located in the center of the device for the chest component can measure the pulse oximeter waveform from the sternum and is integrated with and complemented by an ECG sensor to reduce the number of devices required with additional insights into the vital patterns. A multi-layer signal quality guarantee mechanism, on both the firmware level and on the software analysis level, is applied by leveraging the two-component configuration. In some implementations, the signal quality is traced in real-time and a forced calibration is implemented via the synchronization setups of the two patches to further improve the signal quality. Moreover, additional classifications of sleep apneas can be achieved in comparison with conventional home-based systems due to the chest and abdomen configuration and algorithms. The data analytics is automated and accelerated by the software platform. A respiratory signal recovery algorithm converts the tri-axial accelerator signals into the thoracic and abdominal movement signals that fit those recorded from the respiratory inductance plethysmography.

In particular, the disclosed algorithm advantageously reduces the analysis time. This is a labor-intensive process that takes several hours to manually evaluate for patterns and number of events using other system platforms. The algorithm automatically detects these events, how many events have occurred, and when they occurred. Additionally, the algorithm can distinguish between sleeping and waking conditions.

Other features and advantages are set forth in the following claims.

What is claimed is:

1. A sleep monitoring system comprising:
a first component configured to be coupled to a chest of a subject, the first component comprising
a chest device housing,
a first pair of electrode pads mounted on an underside of the chest device housing,
a first electrocardiogram sensor circuit communicatively coupled to the first pair of electrode pads, and
a photoplethysmogram sensor, wherein the photoplethysmogram sensor includes at least one light source and at least one photodetector mounted on the underside of the chest device housing at a location between the first pair of electrode pads; and
a second component configured to be coupled to an abdomen of the subject, the second component comprising
an abdomen device housing,
a second pair of electrode pads mounted on an underside of the abdomen device housing, and
a second electrocardiogram sensor circuit communicatively coupled to the second pair of electrode pads.

2. The sleep monitoring system of claim 1, wherein the first component is communicatively coupled to the second component by a wired communication interface, wherein the first electrocardiogram sensor circuit is synchronized with the second electrocardiogram sensor circuit by a clock signal transmitted from the second component to the first component via the wired communication interface.

3. The sleep monitoring system of claim 2, wherein the second component further includes a battery, and wherein the system is configured to provide operating power to the first component from the battery through the wired communication interface.

4. The sleep monitoring system of claim 1, wherein the system further includes an electronic controller configured to apply a signal surrogate mechanism to evaluate quality of data signals generated by each of a plurality of measurement units and to calculate a biometric based on different data signals based on a result of the evaluation, wherein the plurality of measurement units includes the first electrocardiogram sensor circuit and the photoplethysmogram sensor.

5. The sleep monitoring system of claim 4, wherein the electronic controller is configured to apply the signal surrogate mechanism by
determining a primary data signal for a first biometric, wherein the primary data signal is generated by a first measurement unit of the plurality of measurement units,
determining a secondary data signal for the first biometric, wherein the secondary data signal is generated by a second measurement unit of the plurality of measurement units,
evaluating a signal quality of the primary data signal,
calculating the first biometric based on the primary data signal in response to determining that the signal quality of the primary data signal satisfies one or more signal quality criterion for the primary data signal,
evaluating a signal quality of the secondary data signal, and
calculating the first biometric based on the secondary data signal in response to determining that the signal quality of the primary data signal does not satisfy the one or more signal quality criterion for the primary data signal and that the signal quality of the secondary data signal satisfies one or more signal quality criterion for the secondary data signal.

6. The sleep monitoring system of claim 5, wherein the electronic controller is further configured to apply the signal surrogate mechanism by not calculating the first biometric in response to determining that the signal quality of the primary data signal does not satisfy the one or more signal quality criterion of the primary data signal and that the signal quality of the secondary data signal does not satisfy the one or more signal quality criterion for the secondary data signal.

7. The sleep monitoring system of claim 5, wherein the primary data signal includes an ECG output signal from at least one selected from the first electrocardiogram sensor circuit and the second electrocardiogram sensor circuit, wherein the secondary data signal includes a PPG output signal from the photoplethysmogram sensor, and wherein the first biometric includes a heart rate metric.

8. The sleep monitoring system of claim 5, wherein the plurality of measurement units further includes at least one inertial measurement unit, wherein the primary data signal includes an IMU output signal from at least one inertial measurement unit, wherein the secondary data signal includes an ECG output signal from at least one selected from the first electrocardiogram sensor circuit and the second electrocardiogram sensor circuit, and wherein the first biometric includes a respiratory effort metric.

9. The sleep monitoring system of claim 1, wherein the first component further includes a first inertial measurement unit configured to monitor movement of the chest device housing due to movements of the chest of the subject, and wherein the second component further includes a second inertial measurement unit configured to monitor movement of the abdomen device housing due to movements of the abdomen of the subject.

10. The sleep monitoring system of claim 9, further comprising an electronic controller configured to monitor a breathing pattern of the subject based at least in part on an output signal of the first inertial measurement unit and an output signal of the second inertial measurement unit.

11. The sleep monitoring system of claim 10, wherein the electronic controller is further configured to:
automatically detect an apnea event based at least in part on disruptions of the breathing pattern of the subject determined based at least in part on the output signal of the monitoring a breathing pattern of the subject based on the output signal of the first inertial measurement unit and the output signal of the second inertial measurement unit.

12. The sleep monitoring system of claim 11, wherein the electronic controller is further configured to:
log the detected apnea event in a memory, wherein the logged apnea event includes a time stamp; and
analyze biometric data corresponding to the logged apnea event based at least in part on the time stamp for the apnea event, wherein the biometric data is determined based at least in part on at least one selected from a group consisting of the first electrocardiogram sensor circuit, the second electrocardiogram sensor circuit, and the photoplethysmogram sensor.

13. The sleep monitoring system of claim 10, wherein the electronic controller is further configured to automatically detect each of a plurality of different types of potential apnea events, wherein the electronic controller is configured to automatically detect each different type of potential apnea event based at least in part on at least one selected from a group consisting of the output signal of the first inertial measurement unit and the output signal of the second inertial measurement unit.

14. The sleep monitoring system of claim 1, wherein the first component further includes an electrical impedance plethysmography circuit, wherein the electrical impedance plethysmograph circuit is communicatively coupled to at least one electrode pad of the first pair of electrode pads and at least one electrode pad of the second pair of electrode pads.

15. The sleep monitoring system of claim 1, wherein the second component further includes a body temperature sensor and a pressure sensor.

16. The sleep monitoring system of claim 1, wherein at least one selected from a group consisting of the first component and the second component includes a memory, wherein the memory is configured to store measurement data from each of a plurality of measurement units of the first component and the second component.

17. The sleep monitoring system of claim 16, wherein the at least one selected from the group consisting of the first component and the second component further includes a wired communication interface that is selectively coupleable to an external computer system, and wherein the external computer system is configured to receive the stored measurement data from the memory in response to the external computer system being coupled to the wired communication interface.

18. The sleep monitoring system of claim 1, further comprising:
a trigger button mounted on an exterior of at least one selected from a group consisting of the first component and the second component; and
at least one electronic controller configured to
detect a user activation of the trigger button, and
log a user-indicated event in response to detecting the user activation of the trigger button, wherein the logged user-indicated event includes a time stamp indicative of a time at which the user activation of the trigger button was detected.

19. The sleep monitoring system of claim 1, wherein the at least one electronic controller is further configured to analyze recorded data corresponding to the user-indicated event based on the time stamp of the logged user-indicated event.

20. A method of calculating a first biometric using a sleep monitoring device, the sleep monitoring device including a first component coupled to a chest of a subject and a second component coupled to an abdomen of the subject, wherein the first component and the second component each include at least one signal measurement unit, the method comprising:
determining a primary data signal for the first biometric, wherein the primary data signal is generated by a first measurement unit of a plurality of measurement units, the plurality of measurement units including the at least one signal measurement unit of the first component and the at least one signal measurement unit of the second component,
determining a secondary data signal for the first biometric, wherein the secondary data signal is generated by a second measurement unit of the plurality of measurement units,
evaluating a signal quality of the primary data signal,
calculating the first biometric based on the primary data signal in response to determining that the signal quality of the primary data signal satisfies one or more signal quality criterion for the primary data signal,
evaluating a signal quality of the secondary data signal, and
calculating the first biometric based on the secondary data signal in response to determining that the signal quality of the primary data signal does not satisfy the one or more signal quality criterion for the primary data signal and that the signal quality of the secondary data signal satisfies one or more signal quality criterion for the secondary data signal.
